(19) United States
(12) Patent Application Publication
(10) Pub. No.: US 2004/0224863 A1
(43) Pub. Date: Nov. 11, 2004

(54) Liquid detersive composition containing sodium cocoyl isethionate

(76) Inventors: James Ziming Sun, Fountain Valley, CA (US); James W. Parr, San Juan Capistrano, CA (US)

Correspondence Address: HENKEL CORPORATION, THE TRIAD, SUITE 200, 2200 RENAISSANCE BLVD., GULPH MILLS, PA, 19406, US

(21) Appl. No.: 10/434,790

(22) Filed: May 8, 2003

(51) Int. Cl. C11D 1/00; A61K 7/50

(52) U.S. Cl. 510/130

(57) ABSTRACT

A cleansing composition comprising an isethionate derivative, a surfactant, and water.

### FIELD OF THE INVENTION

This invention is directed to the field of clear and/or opaque detersive compositions for cleansing hair, skin and the surface of an object.

### BACKGROUND OF THE INVENTION

Owing to its excellent skin compatibility, mildness and emollient properties, sodium cocoyl isethionate (SCI) has been used extensively in personal care products, but its use in water has suffered from solubility problems.

Although certain surfactants can increase the solubility of SCI in aqueous solution, they are often more expensive than common surfactants such as sodium lauryl sulfate (SLS) and sodium laureth sulfate (SLES), which do not efficiently solubilize SCI. For example, an aqueous solution of 10% SLS or SLES solubilizes less than 1% SCI. Making more expensive formulas with specialty surfactants somewhat defeats the advantage of using a relatively inexpensive material like SCI.

### SUMMARY OF THE PREFERRED EMBODIMENTS

The inventive composition contains one or more isethionate derivatives of the formula RCOOCH₂CH₂SO₃X, where R is an alkyl group of from 7 to 21 carbon atoms; preferably R is a straight chain alkyl, and most preferably R is C₁₁H₂₃; and X is ammonium, potassium, magnesium or sodium, preferably sodium (hereinafter, collectively, the “Active”). The Active generally is present in the amount of about 1.0% to about 60.0%, preferably in the amount of about 2.0% to about 50%, and more preferably in the amount of 3.0% to about 45.0%, by total weight of composition. The composition also includes anionic and/or amphoteric surfactants (individually and collectively, the “Surfactant”) where the cationic ions are ammonium or ammonium derivatives, preferably triethanolammonium (TEA), diethanolammonium (DEA) and ammoniummethyl propanol (AMP), or magnesium, such as magnesium coceth sulfate, magnesium coco-sulfate, magnesium glycerophosphate, magnesium laureth-11 carboxylate, magnesium laureth sulfate, magnesium laureth-5 sulfate, magnesium laureth-8 sulfate, magnesium laureth-16 sulfate, magnesium lauryl hydroxypropyl sulfonate, magnesium lauryl sulfate, magnesium methyl cocoyl taurate, magnesium myreth sulfate, magnesium oleth sulfate, magnesium palmitoyl glutamate, magnesium PEG-3 cocamide sulfate, magnesium polyethylene glycol 400 lauryl ether sulfate, magnesium polyethylene glycol (1-4) myristyl ether sulfate, magnesium polyethylene glycol (1-4) oleyl ether sulfate, magnesium polyoxyethylene (1-4) myristyl ether sulfate, magnesium polyoxyethylene (1-4) oleyl ether sulfate. The Surfactant is generally present in the inventive composition in the amount of about 1.0% to about 60.0%, preferably in the amount of about 2.0% to about 50.0%, and more preferably in the amount of 3.0% to about 45.0%, by total weight of composition. The remainder of the composition is water, and all weights are based on the total weight of the composition.

### DETAILS OF THE PREFERRED EMBODIMENTS

The present invention is directed to a composition for making clear or opaque detersive products, preferably personal care products. The inventive composition utilizes, preferably, surfactants, with ammonium and/or ammonium derivative or magnesium, cations, to dissolve the Active in the composition so as to make more economic, much milder and better skin compatibility cosmetic cleansing products.

In general, the composition comprises: (A) from about 1.0% to about 60.0% of the Active; (B) from about 1% to about 60% of the Surfactant; and (c) water, with all weights being based on the weight of the composition.

Preferably other well known ingredients can also be used in the formula for boosting foam, protecting the integrity of composition, increasing viscosity, covering the odor of raw material and increasing the attraction of products. These ingredients include alkylamide MEA or alkylamide DEA, sodium chloride, preservatives, fragrances and colors.

As an example of the Active, sodium cocoyl isethionate may be prepared by reacting an alkali metal isethionate and aliphatic coconut fatty acid (or its acid chlorides).

There are many grades of SCI in the market where the active SCI level varies from 65-85%. The unreacted raw materials are normally impurities. The higher the SCI level, the better the performance and also the higher the cost. SCI used in the preferred embodiments are all SCI grades provided in the market. However, to make a very clear cleansing composition, high SCI level raw material is preferred; to make an opaque cleansing composition, both 85% of SCI and 65% work efficiently.

As an alternative to sodium cocoyl isethionate, an acyl isethionate salt may be used where the acyl group is an alkyl ranging from C₇-C₂₁, and the salt is selected from the group consisting of Na⁺, K⁺, Mg²⁺ and NH₄⁺ and mixtures thereof. The Surfactants include alkyl ether sulfates, alkyl sulfates, alpha-olefin sulfonates, sulfosuccinates, acyl peptides, alkyl phosphates, amphoteric surfactants including acylamphoglycinates and acylamphopropionates, and mixtures thereof.

Anionic surfactants with an ammonium ion include without limitation ammonium C12-15 alkyl sulfate, ammonium capryleth sulfate, ammonium cocomonoglyceride sulfate, ammonium coco-sulfate, ammonium cocoyl isethionate, ammonium cocoyl sarcosinate, ammonium C12-15 pareth sulfate, ammonium C6-16 perfluoroalkylethyl phosphate, ammonium C9-10 perfluoroalkylsulfonate, ammonium dinonyl sulfosuccinate, ammonium dodecylbenzenesulfonate, ammonium laureth-6 carboxylate, ammonium laureth-8 carboxylate, ammonium laureth-2 sulfate, ammonium laureth-3 sulfate, ammonium laureth-5 sulfate, ammonium laureth-7 sulfate, ammonium saureth-9 sulfate, ammonium laureth-12 sulfate, ammonium lauroyl sarcosinate, ammonium lauryl sulfate, ammonium lauryl sulfosuccinate, ammonium myreth sulfate, ammonium myristyl sulfate, ammonium nonoxynol-4 sulfate, ammonium nonoxynol-30 sulfate, ammonium oleate, ammonium palm kernel sulfate, ammonium xylenesulfonate, diammonium lauramido-MEA sulfosuccinate, diammonium lauryl sulfosuccinate, diammonium oleamido PEG-2 sulfosuccinate, and mixtures thereof.

Anionic surfactants with triethanolammonium ion include without limitation TEA-C12-13 alkyl phosphate, TEA-PEG-3 cocamide sulfate, TEA-lauryl sulfate, di-TEA-oleamido PEG-2 sulfosuccinate, TEA-coco-Sulfate, TEA-lauroyl collagen amino acids, TEA-lauroyl keratin amino acids, TEA-undecylenoyl collagen amino acids, TEA-undecylenoyl hydrolyzed Corn Protein, TEA-undecylenoyl hydrolyzed soy protein, TEA-undecylenoyl hydrolyzed wheat protein, TEA-C10-15 alkyl sulfate, TEA-C12-13 alkyl sulfate, TEA-C12-14 alkyl sulfate, TEA-C12-15 alkyl sulfate, TEA-coco-sulfate, TEA-C12-13 pareth-3 sulfate, TEA-dextrin octenylsuccinate, TEA-dimethicone PEG-7 phosphate, TEA-dodecylbenzenesulfonate, TEA-isostearoyl, hydrolyzed collagen, TEA-laneth-5 sulfate, TEA-laureth-4 phosphate, TEA-laureth sulfate, TEA-lauroyl collagen amino acids, TEA-lauroyl keratin amino acids, TEA-lauroyl lactylate, TEA-lauroyl methylaminopropionate, TEA-lauroyl/myristoyl aspartate, TEA-lauroyl sarcosinate, TEA-lauryl phosphate, TEA-myristaminopropionate, TEA-oleoyl hydrolyzed collagen, TEA-oleoyl sarcosinate, TEA-oleyl sulfate, TEA-palm kernel sarcosinate, TEA-PEG-50 hydrogenated castor oil succinate, TEA-phenylbenzimidazole sulfonate, TEA-tridecylbenzenesulfonate, TEA-coco hydrolyzed animal protein, TEA-coco hydrolyzed protein, TEA-cocoyl hydrolyzed animal protein, TEA-hydrogenated tallow glutamate, TEA-lauroyl animal collagen amino acids, TEA-lauroyl animal keratin amino acids, TEA-N-lauryl, myristyl 60-aminopropionate, TEA-monooleamidosulfosuccinate, TEA-myristoyl hydrolyzed animal protein, TEA-oleamido PEG-2 sulfosuccinate, TEA-oleoyl hydrolyzed animal protein, TEA-undecylenoyl hydrolyzed animal protein and mixtures thereof.

Anionic surfactants with diethanolammonium ion include without limitation DEA-C12-13 alkyl sulfate, DEA-C12-15 alkyl sulfate, DEA-ceteareth-2 phosphate, DEA-cetyl phosphate, DEA-cetyl sulfate, DEA-cocoamphodipropionate, DEA-C12-13 pareth-3 sulfate, DEA-C8-18 perfluoroalkylethyl phosphate, DEA dodecylbenzenesulfonate, DEA-hydrolyzed lecithin, DEA-lauraminopropionate, DEA-laureth sulfate, DEA-lauryl sulfate, DEA-methyl myristate sulfonate, DEA-myreth sulfate, DEA-myristyl sulfate, DEA-oleth-3 phosphate, DEA-oleth-5 phosphate, DEA-oleth-10 phosphate, DEA-oleth-20 phosphate, DEA-polyperfluoroethoxymethoxy PEG-2 phosphate, and mixtures thereof.

Anionic surfactants with ammoniummethyl propanol ion include without limitation AM-P-alkyl sulfate, AMP-alkyl ether sulfate, AMP-lauryl phosphate, AMP-isostearoyl hydrolyzed collagen, AMP-isostearoyl hydrolyzed elastin, AMP-isostearoyl hydrolyzed keratin, AMP-isostearoyl hydrolyzed soy protein, AMP-isostearoyl hydrolyzed wheat protein, AMP-isostearoyl wheat/corn/soy amino acids, and mixtures thereof.

The invention is further illustrated by the following exemplar formulations, which are intended as illustrations only and not as a limitation to the scope of the invention. Example 1 clear economic shampoo formula

| INCI Name | Percentage (%) |
|---|---|
| Ammonium Lauryl Sulfate | 7.00 |
| Ammonium Laureth Sulfate | 5.25 |
| Cocamide MEA | 2.15 |
| Polyquternium-10 | 0.25 |
| Sodium Cocoyl Isethionate | 10.0 |
| Linoleamidopropyl PG-dimonium | 0.3 |
| Chloride Phosphate | |
| Viscosity adjustor | q.s |
| Preservatives | q.s. |
| Fragrance | q.s. |
| D.I. water | To 100% |

### Example 2

### Clear Bodywash Formula

| INCI Name | Percentage (%) |
|---|---|
| TEA-Cocoyl Sarcosinate | 15.00 |
| Cocamide MEA | 2.15 |
| Sodium Cocoyl Isethionate | 15.0 |
| Linoleamidopropyl PG-dimonium | 0.5 |
| Chloride Phosphate | |
| Polyquaternium-7 | 0.4 |
| Viscosity adjustor | q.s |
| Preservatives | q.s. |
| Fragrance | q.s. |
| D.I. water | To 100% |

### Example 3

### Clear Mild Facial Cleanser

| INCI Name | Percentage (%) |
|---|---|
| TEA-C12-13 Alkyl Phosphate | 12.00 |
| Cocamide MEA | 2.00 |
| Sodium Cocoyl Isethionate | 10.0 |
| Linoleamidopropyl PG-dimonium | 0.6 |
| Chloride Phosphate | |
| Polyquaternium-7 | 0.3 |
| Viscosity adjustor¹ | q.s |
| Preservatives | q.s. |
| Fragrance | q.s. |
| D.I. water | To 100% |

¹Viscosity adjustors include without limitation ammonium chloride, sodium chloride, Peg-6000 distearate, Peg-120 methyl glucose dioleate, PEG-150 Pentaerythrityl Tetrastearate, natural gums, cellulose derivatives, carbomers, inorganic bentonites and mixtures thereof.

### Example 4

### Detersive Formula

| INCI Name | Percentage (%) |
|---|---|
| Ammonium Laureth Sulfate | 30. |
| Cocamide MEA | 2.5 |
| Sodium Cocoyl Isethionate | 30.0 |
| Hydroxypropyl Methylcellulose | 0.25 |
| Sunflower oil | 10 |
| Viscosity adjustor | q.s |
| Preservatives | q.s. |
| Fragrance | q.s. |
| D.I. water | To 100% |

### Example 5

### Detersive Formula

| INCI Name | Percentage (%) |
|---|---|
| DEA-Lauraminopropionate | 40. |
| Cocamide MEA | 2.15 |
| Sodium Cocoyl Isethionate | 20.0 |
| Polyethylene (beads) | 7.0 |
| Cetyl alcohol | 2.0 |
| Viscosity adjustor | q.s |
| Preservatives | q.s. |
| Fragrance | q.s. |
| D.I. water | To 100% |

### Example 6

### Detersive Formula

| INCI Name | Percentage (%) |
|---|---|
| Ammonium cocoyl isethionate (25% active) | 50.0 |
| Cocamide MEA | 5.15 |
| Sodium Cocoyl Isethionate | 20.0 |
| Dimethicone | 3.0 |
| Viscosity adjustor | q.s |
| Preservatives | q.s. |
| Fragrance | q.s. |
| D.I. water | To 100% |

### Example 7

### Detersive Formula

| INCI Name | Percentage (%) |
|---|---|
| AMP Laureth Sulfate | 15.5 |
| Cocamide MEA | 5.15 |
| Sodium Cocoyl Isethionate | 20.0 |
| Polyquaternium-11 | 0.3 |
| Viscosity adjustor | q.s |
| Preservatives | q.s. |
| Fragrance | q.s. |
| D.I. water | To 100% |

### Example 8

| INCI Name | Percentage (%) |
|---|---|
| TEA-lauroyl methylaminopropionate | 20. |
| Cocamide MEA | 5.15 |
| Sodium cocoyl isethionate | 30.0 |
| Polyquaternium-55 | 0.35 |
| Viscosity adjustor | q.s |
| Preservatives | q.s. |
| Fragrance | q.s. |
| D.I. water | To 100% |

### Example 9

### Clear Shower Gel

| INCI Name | Percentage (%) |
|---|---|
| Sodium laureth sulfate | 20.00 |
| Cocamide MEA | 2.15 |
| Sodium cocoyl isethionate | 15.0 |
| Linoleamidopropyl PG-dimonium | 0.6 |
| Chloride Phosphate | |
| Polyquaternium-7 | 0.4 |
| Viscosity adjustor | q.s |
| Preservatives | q.s. |
| Fragrance | q.s. |
| D.I. water | To 100% |

### Example 10

### Gentle Facial Cleanser

| INCI Name | Percentage (%) |
|---|---|
| magnesium coceth sulfate | 20.00 |
| Cocamide MEA | 2.35 |
| Sodium cocoyl isethionate | 20.0 |
| Linoleamidopropyl PG-dimonium | 0.5 |
| Chloride Phosphate | |
| Polyquaternium-7 | 0.4 |
| Viscosity adjustor | q.s |
| Preservatives | q.s. |
| Fragrance | q.s. |
| D.I. water | To 100% |

1. A detersive composition comprising;
A. from about 1 to about 60% by weight of sodium cocoyl isethionate;
B. from about 1 to about 60% by weight of an anionic and/or amphoteric surfactant where the cationic ion is selected from the group consisting of ammonium or ammonium derivates, and mixtures thereof; and
C. water, all weights being based on the weight of the composition.

2. The composition of claim 1 containing from about 2 to about 50% by weight of sodium coccyl isethionate.

3. The composition of claim 1 wherein the anionic surfactant is an ammonium salt.

4. The composition of claim 3 wherein the surfactant is selected from the group consisting of ammonium alkyl ether sulfates, ammonium alkyl sulfates, ammonium alpha-olefin sulfonates, ammonium alkyl sulfosuccinates, ammonium acyl peptides, ammonium alkyl phosphates, amphoteric surfactants and mixtures thereof.

5. The composition of claim 1 wherein the anionic surfactant is an triethanolammonium salt.

6. The composition of claim 5 wherein the surfactant is selected from the group consisting of triethanolammonium alkyl ether sulfates, triethanolammonium alkyl sulfates, triethanolammonium α-Olefin sulfonates, triethanolammonium alky sulfosuccinates, triethanolammonium acyl peptides, triethanolammonium alkyl phosphates, amphoteric surfactants and mixtures thereof.

7. The composition of claim 1 wherein the anionic surfactant is a diethapolammonium salt.

8. The composition of claim 7 wherein the surfactant is selected from the group consisting of diethanolammonium alkyl ether sulfates, diethanolammonium alkyl sulfates, diethanolammonium α-Olefin sulfates, diethanolammonium alkyl sulfosuccinates, diethanolammonium acyl peptides, diethanolammonium alkyl phosphates, amphoteric surfactants and mixtures thereof.

9. The composition of claim 1 wherein the anionic surfactant is an ammoniumethyl propanol salt.

10. The composition of claim 9 wherein the surfactant is selected from the group consisting of ammoniumethyl propanol alkyl ether sulfates, ammoniumethyl propanol alkyl sulfates, ammoniumethyl propanol, alphaolefin sulfonates, ammoniumethyl propanol alkyl sulfosuccinates, ammoniummethyl propanol acyl peptides, ammoniummethyl propanol alkyl phosphates, amphoteric surfactants and mixtures thereof.

11. The composition of claim 1 wherein the sodium cocoyl isethionate active level ranges from 65% to 85%.

12. A composition comprising one or more isethionate derivatives of the formula RCOOCH₂CH₂SO₃X, where R is an alkyl group of from 7 to 21 carbon atoms and X is selected from the group consisting of ammonium, potassium, magnesium, sodium, or mixtures thereof; and an anionic and/or amphoteric surfactants where the cationic ions are selected from the group consisting of ammonium or ammonium derivatives, magnesium, and mixtures thereof.

13. The composition according to claim 12 wherein the surfactant is selected from the group consisting of magnesium coceth sulfate, magnesium coco-sulfate, magnesium glycerophosphate, magnesium laureth-11 carboxylate, magnesium laureth sulfate, magnesium laureth-5 sulfate, magnesium laureth-8 sulfate, magnesium laureth-16 sulfate, magnesium lauryl hydroxypropyl sulfonate, magnesium lauryl sulfate, magnesium methyl cocoyl taurate, magnesium myreth sulfate, magnesium oleth sulfate, magnesium palmitoyl glutamate, magnesium PEG-3 cocamide sulfate, magnesium polyethylene glycol 400 lauryl ether sulfate, magnesium polyethylene glycol (1-4) myristyl ether sulfate, magnesium polyethylene glycol (1-4) oleyl ether sulfate, magnesium polyoxyethylene (1-4) myristyl ether sulfate, magnesium polyoxyethylene (1-4) oleyl ether sulfate, or mixtures thereof.

14. The composition of claim 12 wherein R is a straight chain alkyl.

15. The composition of claim 12 where in the number of carbon atoms in R ranges from 7 to 21.

16. The composition of claim 12 where the R is C₁₁H₂₃.

17. The composition of claim 12 where X is sodium.

18. The composition of claim 12 where the composition includes sodium cocoyl isethionate.

19. The composition of claim 12 where the surfactant is present in the amount or about 1.0% to about 60.0%, by total weight of composition.

20. The composition of claim 12 where the surfactant is present in the amount of about 2.0% to about 50.0%, by total weight of composition.

21. The composition of claim 12 where the surfactant is present in the amount of about 3.0% to about 45.0%, by total weight of composition.

22. The composition of claim 12 where the isethionate derivative is present in the amount of about 1.0% to about 60.0%, by total weight of the composition.

23. The composition of claim 12 where the isethionate derivative is present in the amount of about 2.0% to about 50.0%, by total weight of the composition.

24. The composition of claim 12 where the isethionate derivative is present in the amount of about 3.0% to about 45.0%, by total weight of the composition.

25. A composition including an acyl isethionate salt, wherein the acyl group is an alkyl ranging from C₇-C₂₁, and the salt is selected from the group consisting of Na⁺, K⁺, Mg²⁺ and NH₄⁺ and mixtures thereof; and a surfactant selected from the group consisting of alkyl ether sulfates, alkyl sulfates, alpha-olefin sulfonates, sulfosuccinates, acyl peptides, alkyl phosphates, amphoteric surfactants and mixtures thereof.

26. The composition of claim 25 where the surfactant is selected from the group consisting of acylamphoglycinates, acylamphopropionates, and mixtures thereof.

27. The composition of claim 12 containing from about 2 to about 50% by weight of sodium coccyl isethionate.

28. The composition of claim 12 wherein the anionic surfactant is an ammonium salt.

29. The composition of claim 28 wherein the surfactant is selected from the group consisting of ammonium alkyl ether sulfates, ammonium alkyl sulfates, ammonium alpha-olefin sulfonates, ammonium alkyl sulfosuccinates, ammonium acyl peptides, ammonium alkyl phosphates, amphoteric surfactants and mixtures thereof.

30. The composition of claim 12 wherein the anionic surfactant is an triethanolammonium salt.

31. The composition of claim 30 wherein the surfactant is selected from the group consisting of triethanolammonium alkyl ether sulfates, triethanolammonium alkyl sulfates, triethanolammonium alpha-olefin sulfonates, triethanolammonium alkyl sulfosuccinates, triethanolammonium acyl peptides, triethanolammonium alkyl phosphates, amphoteric surfactants and mixtures thereof.

32. The composition of claim 12 wherein the anionic surfactant is a diethanolammonium salt.

33. The composition of claim 32 wherein the surfactant is selected from the group consisting of diethanolammonium alkyl ether sulfates, diethanolammonium alkyl sulfates, diethanolammonium alpha-olefin sulfonates, diethanolammonium alkyl sulfosuccinates, diethanolammonium acyl peptides, diethanolammonium alkyl phosphates, amphoteric surfactants and mixtures thereof.

34. The composition of claim 12 wherein the anionic surfactant is an ammoniummethyl propanol salt.

35. The composition of claim 34 wherein the surfactant is selected from the group consisting of ammoniummethyl propanol alkyl ether sulfates, ammonium ethyl propanol alkyl sulfates, ammoniummethyl propanol alpha-olefin sulfonates, ammoniummethyl propanol alkyl sulfosuccinates, ammoniummethyl propanol acyl peptides, ammoniummethyl propanol alkyl phosphates, amphoteric surfactants and mixtures thereof.

36. The composition of claim, 12 where the sodium cocoyl isethionate active level ranges front 65% to 85%.

37. A method of manufacturing a detersive composition comprising the step of mixing one or more isethionate derivatives of the formula RCOOCH₂CH₂SO₃X, where R is an alkyl group of from 7 to 21 carbon atoms and X is selected from the group consisting of ammonium, potassium, magnesium, sodium, or mixtures thereof; an anionic and/or amphoteric surfactant where the cationic ions are selected from the group consisting of ammonium or ammonium derivatives, magnesium, and mixtures thereof; and water.

38. A method of cleaning an object comprising the step of applying to the object a composition comprising one or more isethionate derivatives of the formula RCOOCH₂CH₂SO₃X, where B is an alkyl group of from 7 to 21 carbon atoms and X is selected from the group consisting of ammonium, potassium, magnesium, sodium, or mixtures thereof; an anionic and/or amphoteric surfactant where the cationic ions are selected from the group consisting of ammonium or ammonium derivatives, magnesium, and mixtures thereof; and water.
